(19) United States
(12) Patent Application Publication
(10) Pub. No.: US 2016/0262690 A1
(43) Pub. Date: Sep. 15, 2016

(54) METHOD FOR MANAGING SLEEP QUALITY AND APPARATUS UTILIZING THE SAME

(71) Applicant: MediaTek Inc., Hsin-Chu (TW)

(72) Inventors: Tsan-Jieh CHEN, Hsinchu City (TW); Shu-Yu HSU, Taipei City (TW); Chien-Hua HSU, Zhubei City (TW)

(21) Appl. No.: 14/656,487

(22) Filed: Mar. 12, 2015

(51) Int. Cl. A61B 5/00; A61B 5/0205

(52) U.S. Cl. CPC A61B 5/4815; A61B 5/02055; A61B 5/4812; A61B 5/7405; A61B 5/742; A61B 5/7455; A61B 5/02438

(57) ABSTRACT

A sleep quality management apparatus includes a sensor module and a processing unit. The sensor module is configured to provide a heart rate signal and a skin conductance signal. The processing unit is coupled to the sensor module. The processing unit is configured to determine a sleep stage and a stress level according to the heart rate signal and the skin conductance signal so as to identify a stressful dream occurrence. The stressful dream occurrence is identified when the sleep stage corresponds to a rapid eye movement (REM) stage and the stress level corresponds to a stressful state.

### BACKGROUND OF THE INVENTION

1. Field of the Invention

The invention relates generally to personal health devices, computing devices, and methods for collecting personal health data, and more particularly, to personal health devices, computing devices, and methods for sleep quality management.

2. Description of the Related Art

Sleep is critical to health and poor sleep quality is a principal contributor to many health problems. Typically an individual has four to six sleep cycles per night, each between 60 and 120 minutes in length and comprising different proportions of rapid eye movement (REM) stage and non-REM stage (that is further divided into stages N1, N2 and N3). The sequence of sleep stages (non-REM stages N1, N2, N3 and REM stage) during an overnight sleep is sometimes interrupted with brief periods of wakefulness. The lighter non-REM stages appear first (stages N1 and N2), and often alternate with brief episodes of wakefulness before the deeper non-REM stage is entered (stage N3). The REM stage appears at around 90 minute intervals. As the night progresses the REM stages become longer and non-REM stages become both shorter and lighter. A physiological signal such as heart rate has been used to determine a subject's sleep stages.

REM stage is essential to our minds for processing and consolidating emotions, memories and stress. Most dreaming occurs during REM stage, although it can happen during other sleep stages as well. Bad dreams such as nightmares deteriorate sleep quality. Known methods of detecting bad dreams include the analysis of Electroencephalography (EEG) signals based on the proportion between the deeper non-REM stage and the lighter non-REM stages.

### BRIEF SUMMARY OF THE INVENTION

Sleep quality management apparatus, processing units, and methods for sleep quality management are provided. An exemplary embodiment of the sleep quality management apparatus comprises a sensor module and a processing unit. The sensor module is configured to provide a heart rate signal and a skin conductance signal. The processing unit is coupled to the sensor module and configured to determine a sleep stage and a stress level according to the heart rate signal and the skin conductance signal so as to identify a stressful dream occurrence. The stressful dream occurrence is identified when the sleep stage corresponds to a rapid eye movement (REM) stage and the stress level corresponds to a stressful state.

An exemplary embodiment of the processing unit comprises a sleep stage classifier, a stress level detector and a stressful dream identifier. The sleep stage classifier is configured to determine a sleep stage according to a heart rate signal and a sleep stage classification model. The stress level detector is configured to determine a stress level according to a skin conductance signal and a stress level classification model. The stressful dream identifier is configured to identify a stressful dream occurrence according to the sleep stage and the stress level. The stressful dream occurrence is identified when the sleep stage corresponds to a rapid eye movement (REM) stage and the stress level corresponds to a stressful state.

An exemplary embodiment of the method for sleep quality management executed by an apparatus comprising a sensor module and a processing unit is provided. The sleep quality management method comprises the steps of: determining a sleep stage according to a heart rate signal; determining a stress level according to a skin conductance signal; and identifying a stressful dream occurrence according to the sleep stage and the stress level, wherein the stressful dream occurrence is identified when the sleep stage corresponds to a rapid eye movement (REM) stage and the stress level corresponds to a stressful state.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1A is an exemplary block diagram of a sleep quality management apparatus according to an embodiment of the invention;

FIG. 1B is an exemplary block diagram of a sleep quality management apparatus according to another embodiment of the invention;

FIG. 2 is a block diagram of a sleep quality management apparatus according to an embodiment of the invention;

FIG. 3A shows a schematic of a sleep stage classifier according to an embodiment of the invention;

FIG. 3B shows a signal processing flow for determining sleep stage according to an embodiment of the invention;

FIG. 4A shows a schematic of a stress level detector according to an embodiment of the invention;

FIG. 4B shows a signal processing flow for determining stress level according to an embodiment of the invention;

FIG. 5 shows another signal processing flow for determining sleep stage according to an embodiment of the invention;

FIG. 6 shows another signal processing flow for determining stress level according to another embodiment of the invention;

FIGS. 7A and 7B show power saving implementations for stressful dream detection according to some embodiments of the invention;

FIGS. 8A and 8B show control signals of stressful dream detection for power saving according to some other embodiments of the invention;

FIG. 9 shows a stressful dream detection technique for power saving according to still another embodiment of the invention;

FIG. 10A and FIG. 10B portray an example model showing a wearable device for sleep quality management according to an embodiment of the invention;

FIG. 11 is a flow chart illustrating a method for sleep quality management according to an embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

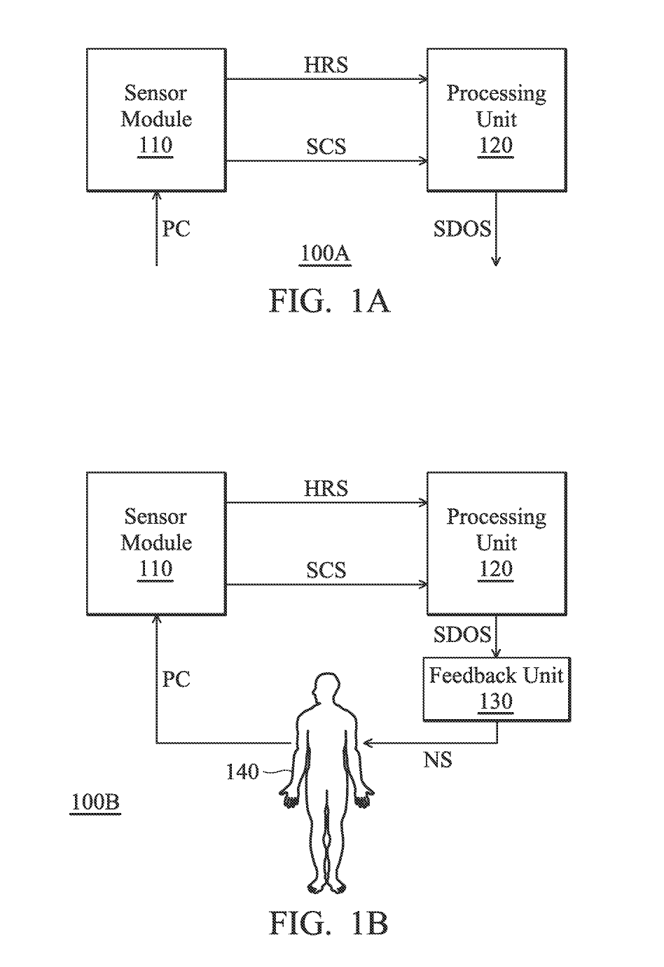

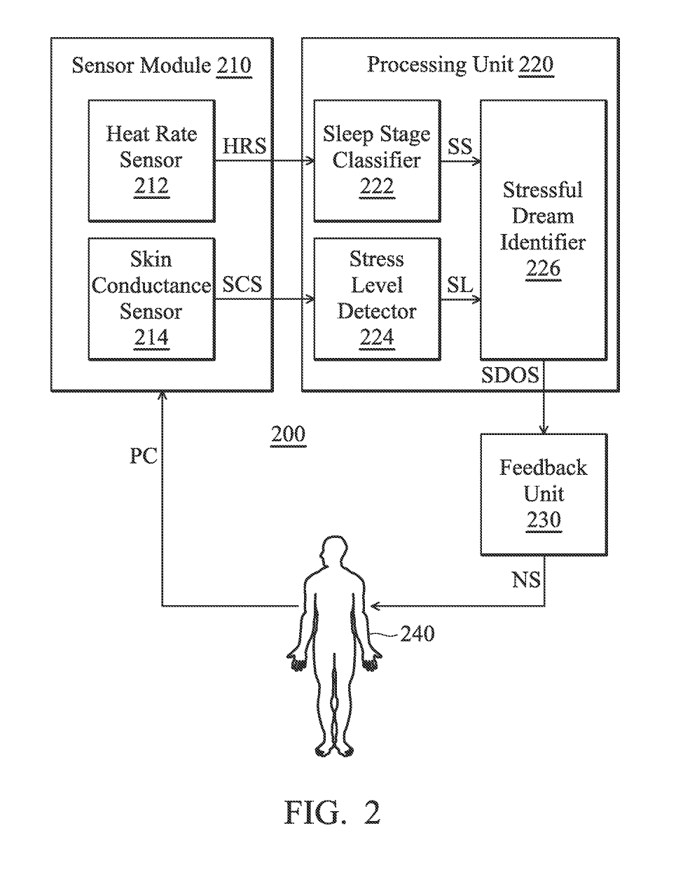

FIG. 1A is an exemplary block diagram of a sleep quality management apparatus according to an embodiment of the invention. The sleep quality management apparatus 100A comprises the sensor module 110 and the processing unit 120. The sensor module 110 is configured to provide the heart rate signal HRS and the skin conductance signal SCS. The input of the sensor module 110 is the physiological characteristics PC, which may be any physiological characteristics suitable for providing information regarding the heart rate signal HRS and the skin conductance signal SCS. The physiological characteristics PC may include, but are not limited to, heart rate, skin conductance, temperature and motion of a human body. The processing unit 120 is coupled to the sensor module 110 and is configured to determine a sleep stage and a stress level according to the heart rate signal HRS and the skin conductance signal SCS so as to identify a stressful dream occurrence. The stressful dream occurrence refers to a physiological state where a person is in a rapid eye movement (REM) stage and the person is under stress. Thus, the stressful dream occurrence is identified when the sleep stage corresponds to the REM stage and the stress level corresponds to a stressful state. The stressful state generally refers to a physiological state during which a user is under quite some stress. More detailed treatment regarding the stressful state will be introduced later. The output of the processing unit 120 is the stressful dream occurrence signal SDOS. The stressful dream occurrence signal SDOS may be a 1-bit signal, which is set to 1′b1 when the stressful dream occurrence is identified and set to 1′b0 when the stressful dream occurrence is not identified.

Note that the heart rate signal HRS may refer to any heart-related physiological signal, from which any heart related physiological information including, but not limited to, heart beats, heart rate (heart beats per minute), and heart rate variability (HRV) may be acquired. HRV refers to the variability of the time interval between heartbeats and is a reflection of an individual's current health status.

FIG. 1B is an exemplary block diagram of a sleep quality management apparatus according to another embodiment of the invention. The sleep quality management apparatus 100B comprises the sensor module 110, the processing unit 120 and the feedback unit 130. The sensor module 110 can be wearable on the user (human body) 140, and the physiological characteristics PC are acquired from the user 140. The difference between FIG. 1A and FIG. 1B is that a feedback unit 130 is added. The feedback unit 130 is coupled to the processing unit 120 and is configured to generate the notification signal NS when the stressful dream occurrence is identified. In one embodiment, the stressful dream occurrence signal SDOS is set to 1′b1 when the stressful dream occurrence is identified; the feedback unit 130 generates the notification signal NS upon “seeing” the stressful dream occurrence signal SDOS is 1′b1. In another embodiment of the invention, the notification signal NS is an audio signal, a light signal or a vibration signal, which may be provided to the user 140 as an alarm.

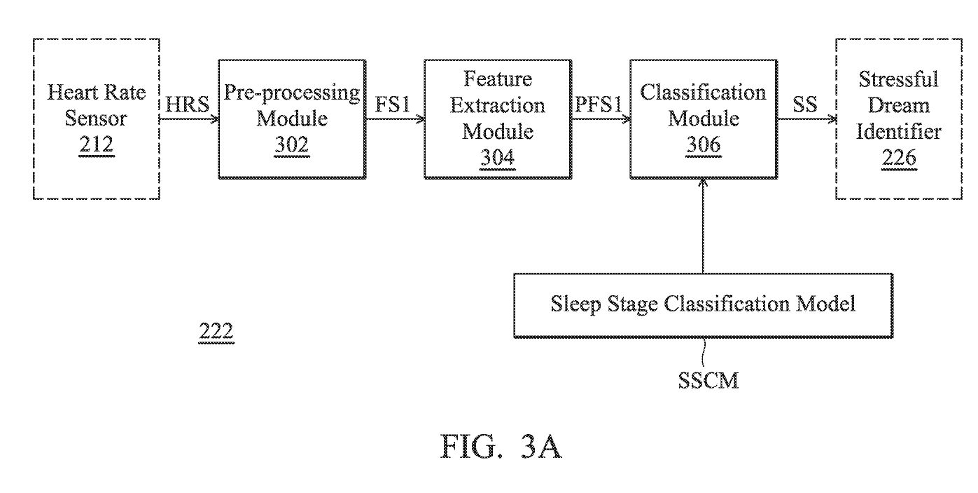

FIG. 2 is a block diagram of a sleep quality management apparatus according to another embodiment of the invention. The sleep quality management apparatus 200 comprises the sensor module 210, the processing unit 220 and the feedback unit 230. The sensor module 210 is configured to provide the heart rate signal HRS and the skin conductance signal SCS according to the physiological characteristics PC of the user 240. The processing unit 220 is coupled to the sensor module 210 and is configured to determine the sleep stage SS and the stress level SL according to the heart rate signal HRS and the skin conductance signal SCS so as to identify the stressful dream occurrence. The stressful dream occurrence is identified when the sleep stage SS corresponds to the REM stage and the stress level SL corresponds to the stressful state. The output of the processing unit 220 is the stressful dream occurrence signal SDOS, which informs the stressful dream occurrence. The feedback unit 230 is coupled to the processing unit 220 and is configured to generate the notification signal NS according to the stressful dream occurrence signal SDOS. The notification signal NS may be at least one of an audio signal, a light signal and a vibration signal, provided to the user 240.

The sensor module 210 comprises the heart rate sensor 212 and the skin conductance sensor 214. The heart rate sensor 212 is configured to provide the heart rate signal HRS and the skin conductance sensor 214 is configured to provide the skin conductance signal SCS. Both the heart rate sensor 212 and the skin conductance sensor 214 may be attached to the user 240.

In one embodiment, the heart rate sensor 212 may be a photoplethysmogram (PPG) sensor. As such, the heart rate signal HRS is a PPG signal. The PPG signal is an optically obtained plethysmogram, a volumetric measurement of an organ. One way to obtain the PPG signal is detecting subcutaneous blood perfusion by shining light through a capillary bed. As arterial pulsations fill the capillary bed, the volumetric changes of the blood vessels modify the absorption, reflection or scattering of the incident light, so the resultant reflected/transmitted light could indicate the timing of cardiovascular events, such as heart rate. Thus, a PPG sensor may include (i) a periodic light source which illuminates the skin, (ii) a photo detector which measures changes in light absorption, and (iii) circuitry determining a user's heart rate from an output of the photo detector. With each cardiac cycle, the heart pumps blood to the periphery. Even though this pressure pulse is somewhat damped by the time it reaches the skin, it is enough to distend the arteries and arterioles in the subcutaneous tissue. The change in volume caused by the pressure pulse is detected by illuminating the skin with the light from a light-emitting diode (LED) and then measuring the amount of light either transmitted or reflected to a photodiode. The PPG signal may be described as a time domain waveform including a DC component and an AC component. The DC component of the signal is attributable to the bulk absorption of the skin tissue, while the AC component is directly attributable to variation in blood volume in the skin caused by the pressure pulse of the cardiac cycle. By analyzing the characteristic of the PPG signal, heart related physiological information such as heart rate can be derived.

The skin conductance sensor 214, or a skin conductance meter, senses the skin conductance from the user 240 to provide the skin conductance signal SCS. The skin conductance refers to the electrical conductance of the skin, which varies depending on the amount of sweat-induced moisture on the skin. Sweat is controlled by the sympathetic nervous system, so the skin conductance is used as an indication of psychological or physiological arousal. If the sympathetic branch of the autonomic nervous system is highly aroused, then sweat gland activity also increases, which in turn increases the skin conductance. In this way, the skin conductance can be used as a measure of emotional and sympathetic responses. Hence, the skin conductance sensor 214 may comprise two electrodes, placed about some distance, to sense the variation of the skin conductance so as to provide the skin conductance signal SCS.

In one embodiment, besides the heart rate sensor 212 and the skin conductance sensor 214, there may be one or more other sensors deployed in the sensor module 210. For instance, a motion sensor or a temperature sensor may be added to function together with the heart rate sensor 212 for getting more accurate heart related physiological information from the user 240. In one embodiment, the sensor module 210 may further comprise a motion sensor configured to detect the motion of the user 240, and a temperature sensor to detect the temperature of the user 240, and the processing unit 220 is configured to determine the sleep stage and the stress level further according to the motion or temperature of the user 240.

The processing unit 220 comprises the sleep stage classifier 222, the stress level detector 224 and the stressful dream identifier 226. The sleep stage classifier 222 is configured to determine the sleep stage SS according to the heart rate signal HRS and a sleep stage classification model. The stress level detector 224 is configured to determine the stress level SL according to the skin conductance signal SCS and a stress level classification model. The stressful dream identifier 226 is configured to identify the stressful dream occurrence according to the sleep stage SS and the stress level SL, and outputs the corresponding stressful dream occurrence signal SDOS. The stressful dream occurrence is identified when the sleep stage SS corresponds to the REM stage and the stress level SL corresponds to the stressful state. In one embodiment, at least some part of the processing unit 220 is implemented by a processor, such as a central processing unit (CPU) or a digital signal processor (DSP), which executes program instructions including machine codes and higher level codes. In another embodiment, the processing unit 220 is implemented by fixed or dedicate hardware logic.

The feedback unit 230 receives the stressful dream occurrence signal SDOS from the stressful dream identifier 226. When the stressful dream occurrence signal SDOS indicates that the stressful dream occurrence is identified, the feedback unit 230 generates the notification signal NS, which may be an audio signal, a light signal or a vibration signal, used to divert the user 240 away from “a stressful dream” state.

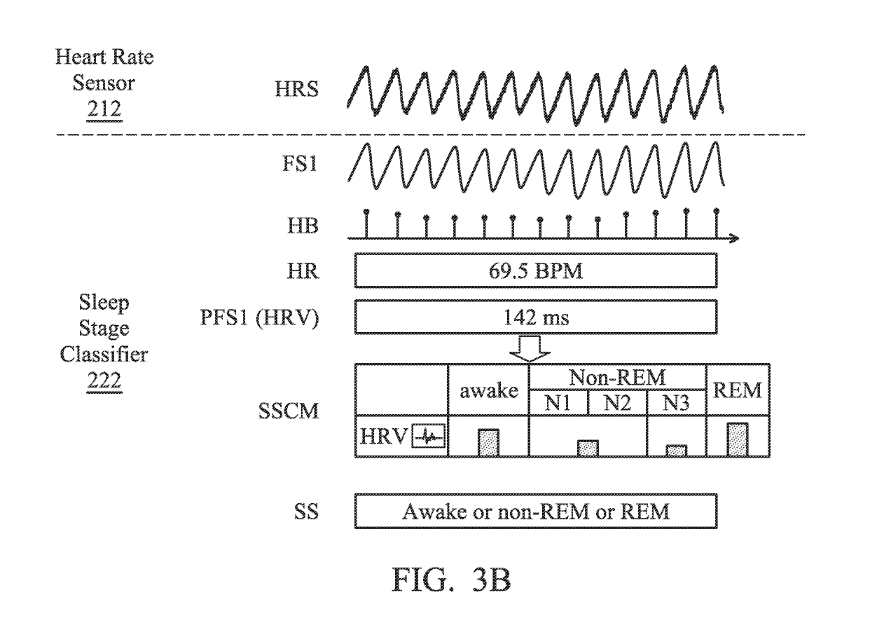

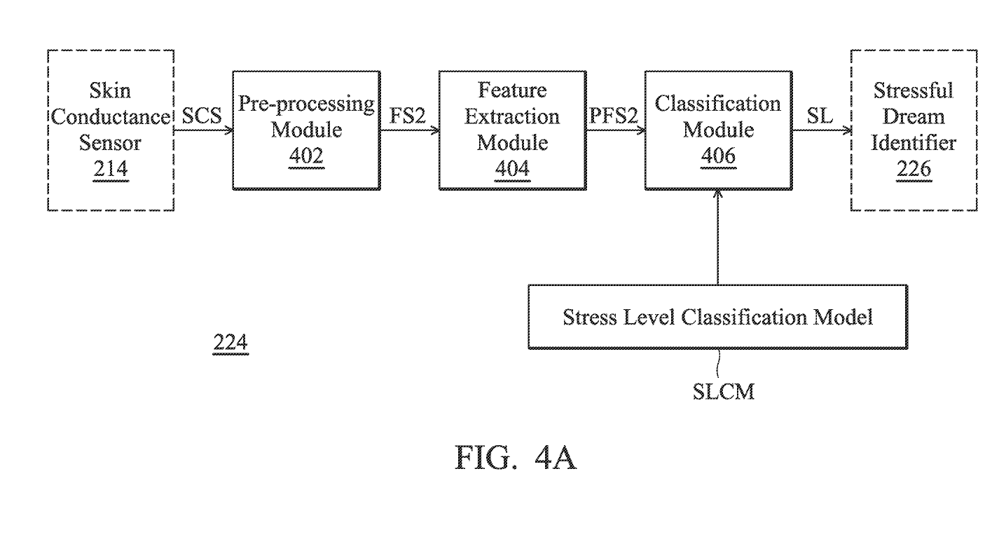

FIG. 3A shows a schematic of a sleep stage classifier according to an embodiment of the invention. The sleep stage classifier 222 comprises the pre-processing module 302, the feature extraction module 304, and the classification module 306. The pre-processing module 302 filters the noise and artifacts inside the heart rate signal HRS from the heart rate sensor 212 to provide the filtered signal FS1. The feature extraction module 304 processes the filtered signal FS1 to derive the physiological feature signal PFS1, which may contain physiological features such as heart beats, heart rate, or HRV. The classification module 306 determines the sleep stage SS according to the physiological feature signal PFS1 and the sleep stage classification model SSCM. Please refer to FIG. 3B for a detailed approach of determining the sleep stage SS.

FIG. 3B shows a signal processing flow for determining a sleep stage according to an embodiment of the invention. Please refer to FIG. 3B in view of FIG. 3A. The heart rate signal HRS from the heart rate sensor 212 is received by the pre-processing module 302. Shown in FIG. 3B, the heart rate signal HRS is to some extent contaminated with some noise or artifacts. The heart rate signal HRS is then filtered by the pre-processing module 302 to provide the filtered signal FS1. The filtered signal FS1 is then processed by the feature extraction module 304 to obtain the heart beats HB. Then, the heart rate HR is obtained by calculating the average time intervals between pairs of consecutive heart beats HB. Here, the heart rate HR is 69.5 beats per minute (BPM). The physiological feature signal PFS1 is further obtained based on the variation of the heart rate HR. Here, the physiological feature signal PFS1 represents the HRV. The feature extraction module 304 then outputs the physiological feature signal PFS1 to the classification module 306, which determines the sleep stage SS according to the physiological feature signal PFS1 and the sleep stage classification model SSCM.

As shown, the classification model SSCM contains different HRV levels associated with different sleep stages. There are five different sleep stages defined in the sleep stage classification model SSCM: awake, non-REM stage (N1, N2, and N3) and the REM stage. Then, the sleep stage SS may be a 3-bit signal to represent the five different sleep stages in the sleep stage classification model SSCM. Typically, HRV during the REM stage is the largest among sleep stages of REM stage, non-REM stage and awake. HRV during the deeper non-REM stage is smaller than that during the lighter non-REM stages. HRV while awake is smaller than that during REM stage but larger than that during lighter non-REM stage. One method to determine the sleep stage SS is by comparing the physiological feature signal PFS1 with the HRV levels of different sleep stages defined in the sleep stage classification model SSCM. Thus, the sleep stage SS, being REM stage, non-REM stage or awake stage, may be determined and output to, say, the stressful dream identifier 226 as shown in FIG. 3A. Of course, more advanced mathematical computation such as correlation between the sleep stage classification model SSCM and the physiological feature signal PFS1 may be adopted instead.

According to another embodiment, the sleep stage classification model SSCM may contain HRV energy components at different frequencies for different sleep stages. Specifically, there can be a low frequency (LF; 0.04-0.15 Hz) part and a high frequency (HF; >0.15 Hz) part. And HRV is known to show an increase in HF components and a decrease in LF components in non-REM stages, while the opposite changes happen during REM stage. Meanwhile, low frequency is reported to show a significant decrease as the sleep stage deepens. With such information in the sleep stage classification model SSCM, through some mathematical manipulations such as those mentioned above, the sleep stage SS may be determined as well.

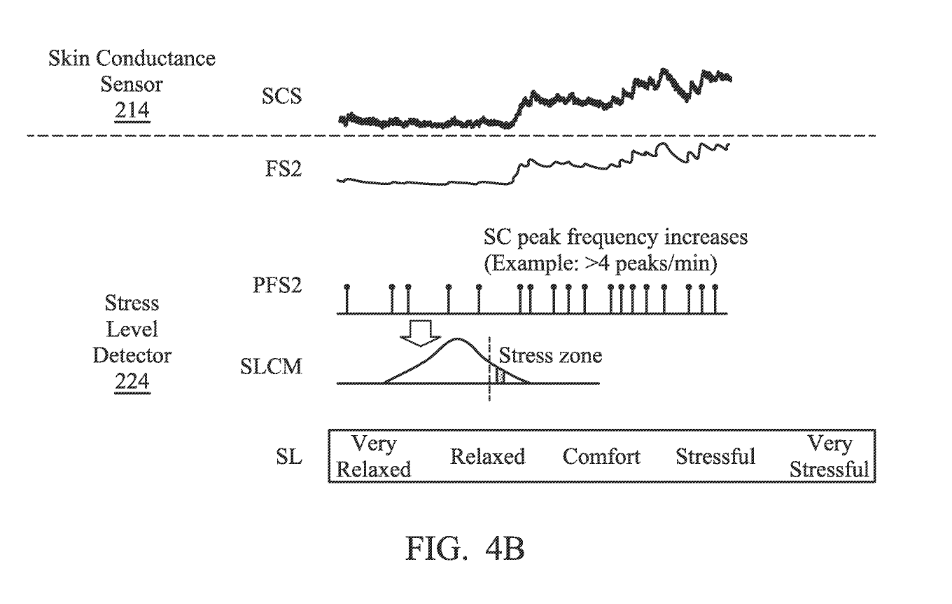

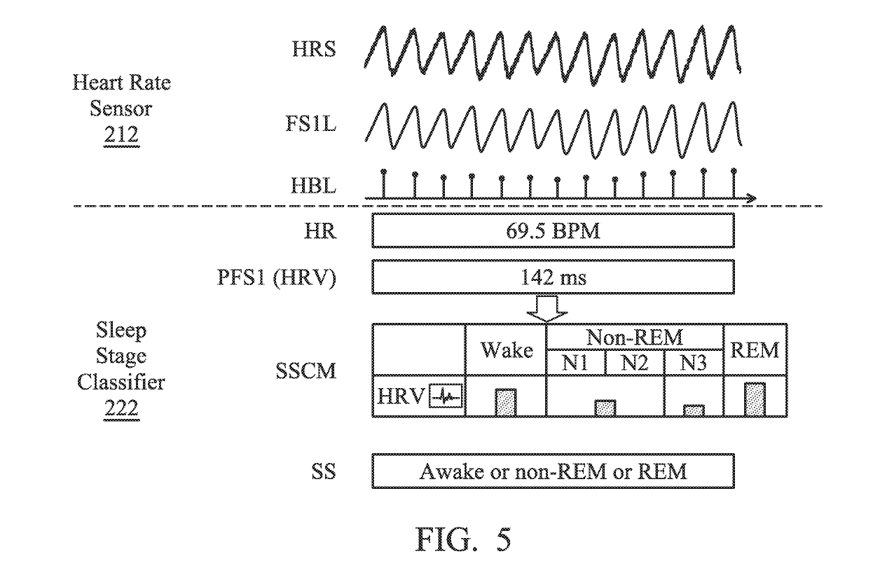

FIG. 4A shows a schematic of a stress level detector according to an embodiment of the invention. The stress level detector 224 comprises the pre-processing module 402, the feature extraction module 404, and the classification module 406. The pre-processing module 402 filters the noise and artifacts inside the skin conductance signal SCS from the skin conductance sensor 214 to provide the filtered signal FS2. The feature extraction module 404 processes the filtered signal FS2 to derive the physiological feature signal PFS2, which may contain physiological features related to the skin conductance signal SCS such as frequency of skin conductance local peak appearance. The classification module 406 determines the stress level SL according to the physiological feature signal PFS2 and the stress level classification model SLCM. Please refer to FIG. 4B for a detailed approach of determining the stress level SL.

Please refer to FIG. 4B in view of FIG. 4A. The stress level detector 224 receives the skin conductance signal SCS, shown as a time domain profile of skin conductance. As shown, the skin conductance signal SCS is to some extent contaminated with some noise or artifacts. The skin conductance signal SCS is then filtered by the pre-processing module 402 for removing the noise or artifacts to provide the filtered signal FS2. The filtered signal FS2 is then processed in the feature extraction module 404 to obtain the physiological feature signal PFS2, which represent the time instants at which the skin conductance signal SCS reaches its local peak values. Then, the physiological feature signal PFS2 is processed in the classification module 406, which compares the rate of the occurrence of skin conductance local peak with the stress level classification model SLCM.

As an example, the stress level classification model SLCM includes distribution of occurrence of skin conductance local peak with respect to different stress levels. Such a distribution may be collected from historical statistics of skin conductance local peak occurrence frequency of a human body. In general, the local peak occurs more frequently as the stress level of a human body increases. Thus, based on the physiological feature signal PFS2 and the stress level classification model SLCM, the stress level SL may be determined through some mathematical techniques analogous to those described regarding the sleep stage classifier 222.

The relationship between the stress level SL and the stressful state is more fully discussed below. Shown in FIG. 4B, the stress level SL output by the stress level detector 224 comes with five levels: “very relaxed”, “relaxed”, “normal”, “stressful” and “very stressful”. As a first example, it may be defined that when the stress level SL is “stressful” or “very stressful”, then the stress level SL corresponds to the stressful state. As a second example, it may be defined only when the stress level SL is “very stressful”, then the stress level SL corresponds to the stressful state. As still another example, such mapping between the stress level SL and the stressful state may be manually set. To cover the five stress levels mentioned above, the stress level SL may be a 3-bit signal. However, as another example, the stress level detector 224 may directly determine whether the stressful state is detected. As such, the stress level SL can be a 1-bit signal. When the stress level SL is set to 1′b1, it means the stressful state is detected. When the stress level SL is set to 1′b0, it means the stressful state is not detected.

Please refer back to FIG. 2 shortly for a detailed description regarding the generation of the stressful dream occurrence signal SDOS. The stressful dream identifier 226 receives the sleep stage SS and the stress level SL from the sleep stage classifier 222 and the stress level detector 224, respectively. Then the stressful dream identifier 226 identifies the stressful dream occurrence according to the sleep stage SS and the stress level SL. The stressful dream occurrence is identified when the sleep stage SS corresponds to the REM stage and the stress level SL corresponds to the stressful state. The stressful dream occurrence signal SDOS may be a 1-bit signal, generated according to the identification of the stressful dream occurrence. In one example, when the stressful dream occurrence is identified, the stressful dream occurrence signal SDOS is set to 1′b1. Otherwise, the stressful dream occurrence signal SDOS is set to 1′b0.

The stressful dream identification discussed above provides some insight for evaluating the sleep quality of a human being. Sleep quality measures “how well” a person sleeps and there are different factors or approaches to evaluate it. For the sleep stages of being awake, N1, N2, N3 and REM defined in the sleep stage classification model SSCM of FIG. 3B, a sleep quality index may be defined as “(FT(N3)+FT(REM)−FT(stressful dream occurrence))/(FT(N1)+FT(N2)+FT(N3)+FT(REM))”, where FT( ) stands for the amount of time in a particular sleep stage a user goes through during sleep. In one embodiment, the sleep quality index may be calculated by the processing unit 220 in FIG. 2. That is, the processing unit 220 is configured to provide the sleep quality index according to a period of the deep sleep stage (N3), a period of the REM stage and a period of the stressful dream occurrence. The sleep quality index may distinguish the REM stage without stress from the REM stage with stress, i.e. stressful dream occurrence. Under the REM stage without stress detected, the sleep quality may be considered good. Under REM stage but with stress detected, the sleep quality may be actually not good. Thus, the sleep quality index may more accurately reflect the true quality of sleep of the user 240. In one embodiment, the sleep quality index is derived by some fixed or dedicate hardware logic in the processing unit 220. In another embodiment, the sleep quality index is calculated by an instruction-based computing module, such as a general purpose processor.

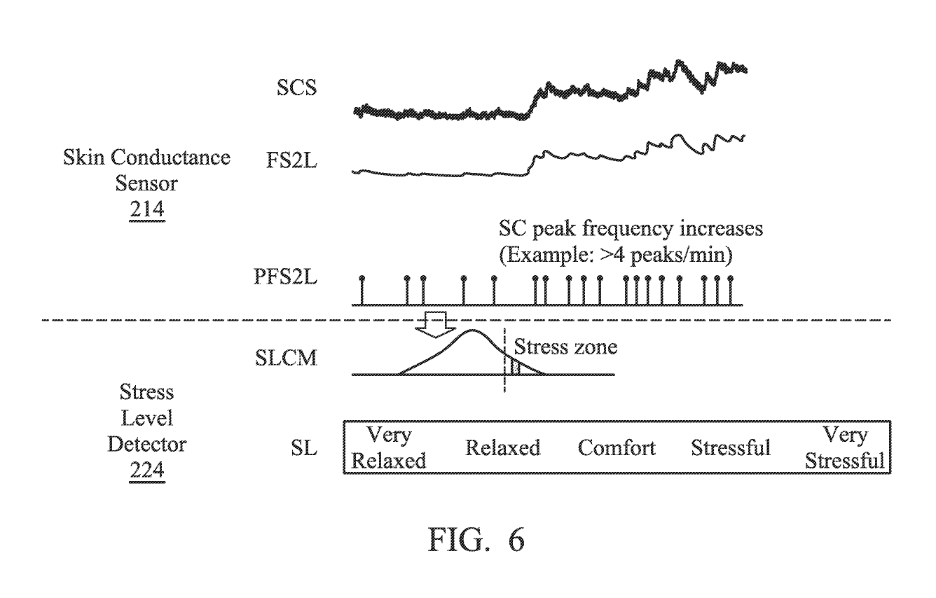

As power becomes a major issue in electronic or medical devices nowadays, some other aspects of the invention according to some other embodiments are shown below. FIG. 5 illustrates another signal processing flow for determining a sleep stage according to another embodiment of the invention. Shown in FIG. 5, the heart rate signal HRS is filtered in the heart rate sensor 212 to derive the filtered signal FS1L. Then, the filtered signal FS1L is processed also in the heart rate sensor 212 to derive the heart beats HBL. This may be realized by putting some analog circuits along with existing sensors in the heart rate sensor module 212. Analog circuits are known to do well for tasks such as filtering and simple arithmetic operations, e.g. finding local peak values in a waveform. Besides, the power consumption in performing such tasks using analog circuits is typically lower as compared with using digital circuits.

In FIG. 5, the input to the sleep stage classifier 222 is the heart beats HBL rather than the heart rate signal HRS. The heart beats HBL may have a sample rate around 1 to 2 samples per second whereas the heart rate signal HRS may have a sample rate around 250 samples per second. This is because a heart beat occurs around every 1 second but to represent the heart rate signal HRS waveform precisely enough, the sampling rate would be above hundreds of samples per second. As the sleep stage classifier 222 may more or less be implemented by digital circuits, a lower input sample rate requires a lower clock speed, which in turn reduces the amount of power consumed. Note that the remaining parts of FIG. 5 can be analogously understood in light of FIG. 3B and shall be omitted here for the sake of brevity.

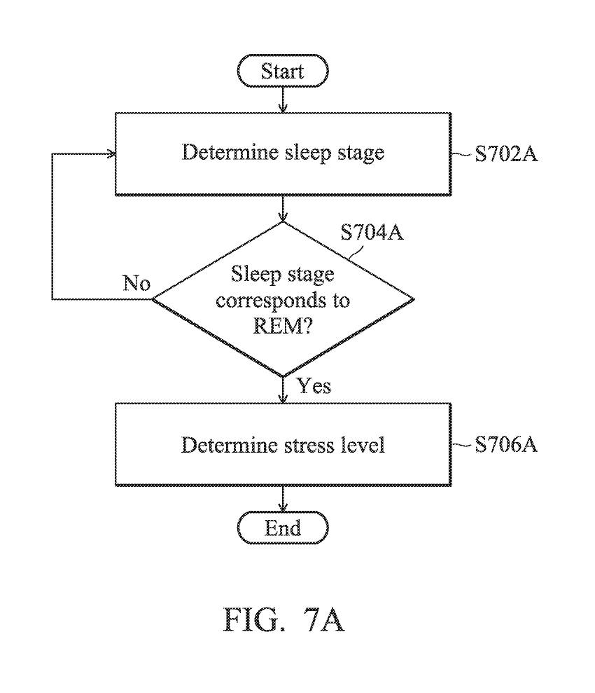

FIG. 6 illustrates another signal processing flow for determining a stress level according to another embodiment of the invention. Shown in FIG. 6, the skin conductance signal SCS is filtered in the skin conductance sensor 214 to derive the filtered signal FS2L. Then, the filtered signal FS2L is processed also in the skin conductance sensor 214 to derive the physiological feature signal PF2L. This may be realized by putting some analog circuits along with existing sensors in the skin conductance sensor 214. Thus, the input to the stress level detector 224 is the physiological feature signal PFS2L rather than the skin conductance signal SCS. It can be suggested that the sample rate of the skin conductance signal SCS is above tens of samples per second while the physiological feature signal PFS2L may have a sample rate below 1 sample per second.

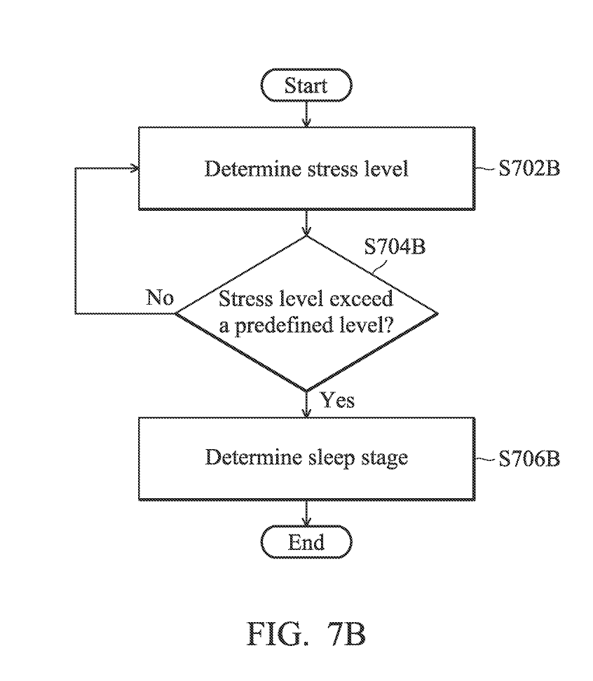

FIGS. 7A and 7B show power saving implementations for stressful dream detection according to some embodiments of the invention. Both figures can be more easily understood when accompanied with FIG. 2. Please refer to FIG. 7A and FIG. 2 first. In step S702A, the sleep stage classifier 222 determines the sleep stage SS according to the heart rate signal HRS and a sleep stage classification model, where the heart rate signal HRS is provided by the heart rate sensor 212. To be more specific, determining the sleep stage may include the two sub-steps below. Firstly, provide an HRV according to the heart rate signal HRS. Secondly, determine the sleep stage SS according to the HRV and the sleep stage classification model. At the same time, the skin conductance sensor 214 as well as the stress level detector 224 may be turned off, i.e., the power consumption dissipated by the skin conductance sensor 214 and the stress level detector 224 may be zero or very little. The skin conductance sensor 214 and the stress level detector 224 are not turned on until the sleep stage is determined to be corresponding to the REM stage.

In step S704A, whether the sleep stage SS corresponds to the REM stage is monitored so that the stress level detector 224 can be activated when the sleep stage SS corresponds to the REM stage. Step S704A may be executed by the sleep stage classifier 222 or the stressful dream identifier 226. Note that step S702A and step S704A may be performed concurrently in practice. In one embodiment, when the sleep stage classifier 222 informs the stressful dream identifier 226 that the sleep stage SS corresponds to the REM stage, a power on signal may be generated by the stressful dream identifier 226 to turn on the power of the skin conductance sensor 214 and the stress level detector 224. Then step S706A is performed and the stress level detector 224 detects the stress level SL. On the other hand, when it is found in step S702A that the sleep stage SS does not correspond to the REM stage, step S706A is not performed so that the stress level detector 224 and the skin conductance sensor 214 remain non-functional. To be reminded, in step S706A, the heart rate sensor 212 and the sleep stage classifier 222 may remain functioning for continual determination of the sleep stage SS.

For another power saving implementation, please then refer to FIG. 7B and FIG. 2. In step S702B, the stress level detector 224 determines the stress level SL according to the skin conductance signal SCS and a sleep stage classification model, where the skin conductance signal SCS is provided by the skin conductance sensor 214. Note that in step S701B, the heart rate sensor 212 as well as the sleep stage classifier 222 is turned off, i.e., the power consumption dissipated by the heart rate sensor 212 and the sleep stage classifier 222 may be zero or very little. The heart rate sensor 212 and the sleep stage classifier 222 are not turned on until the stress level SL is found to exceed a predefined level, e.g. “stressful” defined in FIG. 4B. In step S704B, whether the stress level SL exceeds the predefined level is monitored. Once the stress level SL exceeds the predefined level, the sleep stage classifier 222 is activated for determining the sleep stage SS (step S706B). While the stress level SL does not exceed the predefined level, step S706B is not performed so that the sleep stage classifier 222 and the heart rate sensor 212 remain non-functional. To be reminded, during step S706B, the skin conductance sensor 214 and the stress level detector 224 may remain functioning for continual detection of the stress level SL.

It can be seen that in FIG. 7A the sleep stage classifier 222 is turned on before the stress level detector 224 whereas in FIG. 7B the sleep stage classifier 222 is turned on after the stress level detector 224. Note that this order can be adjusted according to, say, the sleep history of a user. For instance, by default setting, the sleep stage classifier 222 is turned on before the stress level detector 224. That is, the sleep stage classifier 222 is turned on with priority and the stress level detector 224 may be turned off most of the time. After several days of usage, it may be discovered that the user encounters stressful states less frequently than the REM stage. If so, the turn-on priority will be reversed, i.e. the stress level detector 224 is turned on before the sleep stage classifier 222.

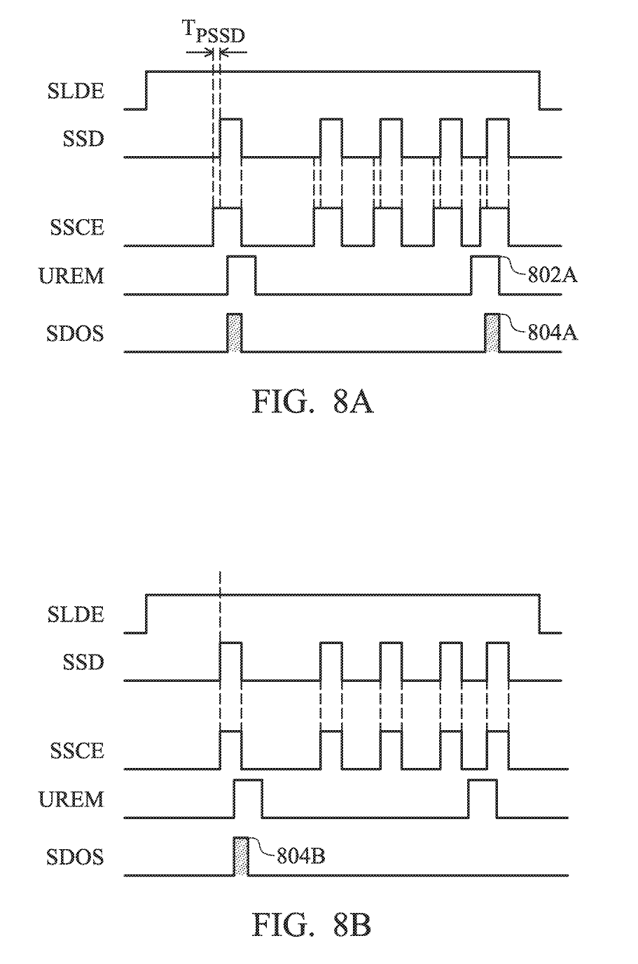

FIGS. 8A and 8B show control signals of stressful dream detection for power saving according to some embodiments of the invention. Please refer to FIG. 8A first accompanied with FIG. 2. The stress level detection enable SLDE, while pulled high, means that the stress level detector 224 remains turned on for detecting whether the stress level SL corresponds to the stressful state. The stressful state detected SSD remains high during the time interval when the stress level SL corresponds to the stressful state and goes low when the stress level SL no longer corresponds to the stressful state. The sleep stage classification enable SSCE, while pulled high, means that the sleep stage classifier 222 is on for determining the sleep stage SS. When the sleep stage classification enable SSCE is low, it means that the sleep stage classifier 222 and the heart rate sensor 212 are turned off. The status UREM represents the time interval during which the user 240 is actually in the REM stage. Note that the status UREM serves only for explanatory purpose and there is no such signal inside the sleep quality management apparatus 200.

Shown in FIG. 8A, the sleep stage classification enable SSCE is asserted shortly before the stressful state detected SSD by the time interval T_{PSSD}. In one embodiment, this is achieved by the stress level detector 224. Once the stress level detector 224 finds the stress level SL is likely to correspond to the stressful state, it pulls the sleep stage classification enable SSCE high. For example, when the stress level detector 224 find the frequency of the appearance of the skin conductance local peak exceeds a predetermined value, it is considered that the stress level SL is likely to correspond to the stressful state. In one embodiment, the sleep stage classification enable SSCE goes low when the stressful state detected SSD goes low. By doing so, the sleep stage classifier 222 switches from off to on before the stress level SL corresponds to the stressful state. Such early turning on the sleep stage classifier 222 may be advantageous for spotting the stressful dream occurrence. Consider the second time interval 802A of status UREM. Since it takes some time for the sleep stage classifier 222 to determine the sleep stage SS, by turning on the sleep stage classifier 222 early, there is enough time for the sleep stage classifier 222 to “capture” the second time interval 802A of status UREM. Then the sleep stage SS may be determined to correspond to the REM stage and the stressful dream occurrence may be identified, as shown in the second time interval 804A of the stressful dream occurrence signal SDOS. From implementation perspective, the T_{PSSD} may be around 300 seconds so as to be beneficial for capturing the period where a user is under the REM stage.

Without the early turning-on technique, i.e. the sleep stage classifier 222 is turned on after the stressful state is detected as shown in FIG. 8B, the second time interval 802A of status UREM in FIG. 8A may not be “seen” long enough by the sleep stage classifier 222. Therefore, the stressful dream occurrence signal SDOS goes high only during the time interval 804B.

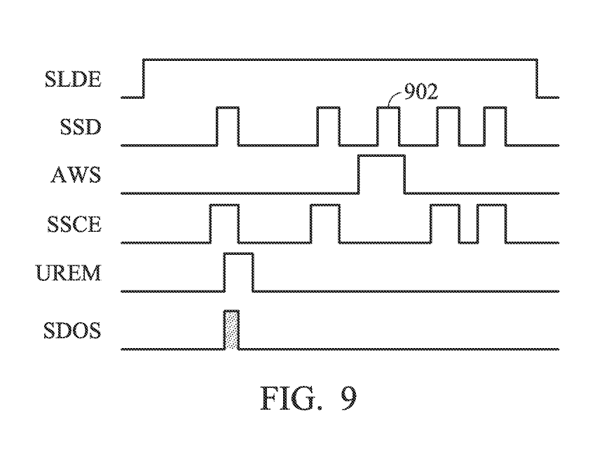

FIG. 9 shows another power-saving technique for detecting stressful dreams according to still another embodiment of the invention. Please refer to FIG. 9 in view of FIG. 2. The major difference between FIG. 9 and FIG. 8A is that the awake signal AWS is added. The awake signal AWS may be provided by an additional motion sensor (not shown in FIG. 2) and is set high when detecting that the user 240 is awake. When the awake signal AWS is high, even though the stress level SL corresponds to the stressful state as shown by 902, the sleep stage classification enable SSCE remains low. Henceforth, the sleep stage classifier 222 is not turned on. In this way, further power-saving may be achieved for the purpose of detecting stressful dream.

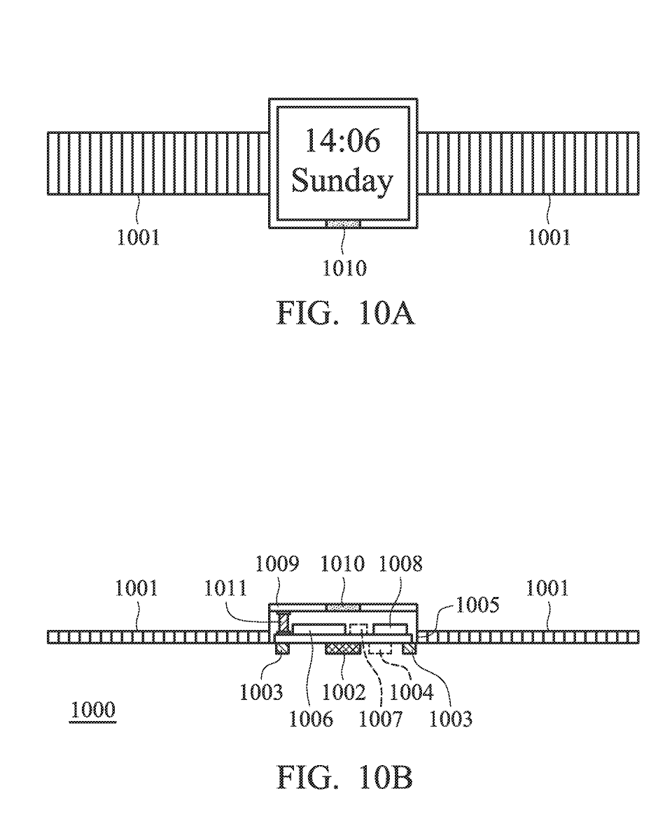

FIG. 10A and FIG. 10B portray an example model showing a wearable device for sleep quality management according to an embodiment of the invention. The wearable device 1000 may be worn on a wrist (watch type) or put on the head (head band type). For the side view, two straps 1001 are shown to be attached to the left and right sides of the wearable device 1000. On the bottom side of the wearable device 1000 reside the heart rate sensor 1002, the two skin conductance electrodes of the skin conductance sensor 1003, and the skin temperature sensor 1004. The skin temperature sensor 1004 is optional. The bottom side is meant to be deployed to contact the skin of a user. Inside the wearable device 1000 are a circuit board 1005, carrying the processing unit 1006, the motion sensor 1007, and the vibrator/buzzer 1008. The vibrator/buzzer 1008 may serve as a feedback unit. The motion sensor 1007 is optional. On top of the wearable device 1000 is a display unit 1009 such as a liquid crystal display (LCD), where a light-emitting diode (LED) 1010 is integrated into one side of the display unit 1009. The LED 1010 may serve as another feedback unit. The display unit 1009 may be coupled to the circuit board 1005 by wire 1011 so that the signal from the processing unit 1006 may be transmitted to the LED 1010 for feedback.

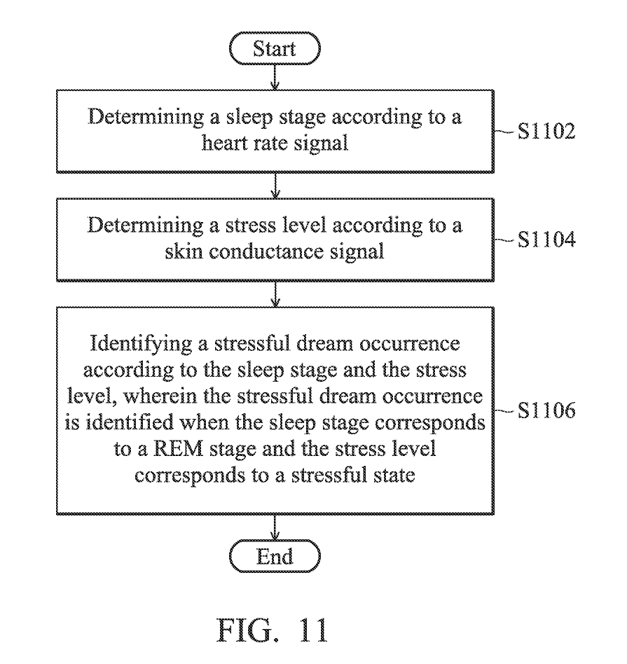

FIG. 11 is a flow chart illustrating a method for sleep quality management executed by an apparatus comprising a sensor module and a processing unit according to an embodiment of the invention. In one embodiment, the method may be performed by the apparatus as shown in FIG. 1A, FIG. 1B or FIG. 2. In step S1102, a sleep stage is determined according to a heart rate signal. Then a stress level is determined according to a skin conductance signal (step S1104). Note the sequence for performing steps S1102 and 1104 have not necessarily been rendered according to any particular sequence. For example, steps S1102 and 1104 may be performed concurrently or in a different order as illustrated in FIG. 11. Next, a stressful dream occurrence is identified according to the sleep stage and the stress level (step S1106), where the stressful dream occurrence is identified when the sleep stage corresponds to the REM stage and the stress level corresponds to a stressful state. Note that, in one embodiment, step S1106 may be carried out concurrently with step S1102 or step S1104. In another embodiment, the steps S1102, S1104 and S1106 may be performed concurrently.

The method according to the embodiments described above may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter.

The functionality discussed herein may be provided using a number of different approaches. For example, in some implementations a processor may be controlled by computer-executable instructions stored in memory so as to provide functionality such as is described herein. In other implementations, such functionality may be provided in the form of an electrical circuit. In yet other implementations, such functionality may be provided by a processor or processors controlled by computer-executable instructions stored in a memory coupled with one or more specially-designed electrical circuits. Various examples of hardware that may be used to implement the concepts outlined herein include, but are not limited to, application specific integrated circuits (ASICs), field-programmable gate arrays (FPGAs), and general-purpose microprocessors coupled with memory that stores executable instructions for controlling the general-purpose microprocessors.

While the invention has been described by way of example and in terms of preferred embodiment, it should be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. A sleep quality management apparatus, comprising:
a sensor module, configured to provide a heart rate signal and a skin conductance signal; and
a processing unit, coupled to the sensor module, configured to determine a sleep stage and a stress level according to the heart rate signal and the skin conductance signal so as to identify a stressful dream occurrence,
wherein the stressful dream occurrence is identified when the sleep stage corresponds to a rapid eye movement (REM) stage and the stress level corresponds to a stressful state.

2. The sleep quality management apparatus as claimed in claim 1, further comprising:
a feedback unit, coupled to the processing unit, configured to generate an audio signal, a light signal or a vibration signal when the stressful dream occurrence is identified.

3. The sleep quality management apparatus as claimed in claim 1, wherein the processing unit comprises a sleep stage classifier, configured to determine the sleep stage according to the heart rate signal and a sleep stage classification model.

4. The sleep quality management apparatus as claimed in claim 3, wherein the sleep stage classifier is triggered when the stress level exceeds a predefined level.

5. The sleep quality management apparatus as claimed in claim 1, wherein the processing unit further comprises a stress level detector, configured to determine the stress level according to the skin conductance signal and a stress level classification model.

6. The sleep quality management apparatus as claimed in claim 5, wherein the stress level detector is triggered when the sleep stage corresponds to the REM stage.

7. The sleep quality management apparatus as claimed in claim 1, wherein the sleep stage further comprises a deep sleep stage, and the processing unit is further configured to provide a sleep quality index according to a period of the deep sleep stage, a period of the REM stage and a period of the stressful dream occurrence.

8. The sleep quality management apparatus as claimed in claim 1, wherein the sensor module comprises:
a heart rate sensor configured to provide the heart rate signal; and
a skin conductance sensor configured to provide the skin conductance signal.

9. The sleep quality management apparatus as claimed in claim 8, wherein the sensor module further comprises a motion sensor configured to provide a motion signal and a temperature sensor configured to provide a temperature signal, and the processing unit is configured to determine the sleep stage and the stress level further according to the motion signal and the temperature signal.

10. The sleep quality management apparatus as claimed in claim 1, wherein the sensor module is wearable on a human body.

11. A processing unit, comprising:
a sleep stage classifier, configured to determine a sleep stage according to a heart rate signal and a sleep stage classification model;
a stress level detector, configured to determine a stress level according to a skin conductance signal and a stress level classification model; and
a stressful dream identifier, configured to identify a stressful dream occurrence according to the sleep stage and stress level,
wherein the stressful dream occurrence is identified when the sleep stage corresponds to a rapid eye movement (REM) stage and the stress level corresponds to a stressful state.

12. The processing unit as claimed in claim 11, wherein the stress level detector is triggered when the sleep stage corresponds to the REM stage.

13. The processing unit as claimed in claim 11, wherein the sleep stage classifier is triggered when the stress level exceeds a predefined level.

14. The sleep quality management apparatus as claimed in claim 11, wherein the sleep stage further comprises a deep sleep stage, and the processing unit further comprises a sleep quality monitor for providing a sleep quality index according to a period of the deep sleep stage, a period of the REM stage and a period of the stressful dream occurrence.

15. A sleep quality management method executed by an apparatus comprising a sensor module and a processing unit, the method comprising:
determining a sleep stage according to a heart rate signal;
determining a stress level according to a skin conductance signal; and
identifying a stressful dream occurrence according to the sleep stage and the stress level,
wherein the stressful dream occurrence is identified when the sleep stage corresponds to a rapid eye movement (REM) stage and the stress level corresponds to a stressful state.

16. The sleep quality management method as claimed in claim 15, further comprising:
generating an audio signal, a light signal or a vibration signal when the stressful dream occurrence is identified.

17. The sleep quality management method as claimed in claim 15, wherein the step of determining the sleep stage comprises:
providing a heart rate variability according to the heart rate signal; and
determining the sleep stage according to the heart rate variability and a sleep stage classification model.

18. The sleep quality management method as claimed in claim 15, wherein the stress level is further determined according to a stress level classification model.

19. The sleep quality management method as claimed in claim 15, wherein the sleep stage further comprises a deep sleep stage, and the method further comprising:
providing a sleep quality index according to a period of the deep sleep stage, a period of the REM stage and a period of the stressful dream occurrence.

20. The sleep quality management method as claimed in claim 15, wherein the sleep stage is further determined according to a temperature signal and a motion signal.
